(19) United States
(12) Patent Application Publication
(10) Pub. No.: US 2010/0062036 A1
(43) Pub. Date: Mar. 11, 2010

(54) Multiblock Copolymers with Shape-Memory Properties

(75) Inventors: Steffen Kelch, Oberengstringen (CH); Andreas Lendlein, Berlin (DE); Yakai Feng, Berlin (DE)

Correspondence Address: COHEN, PONTANI, LIEBERMAN & PAVANE LLP, 551 FIFTH AVENUE, SUITE 1210, NEW YORK, NY, 10176, US

(73) Assignee: GKSS-Forschungszentrum Geesthacht GmbH, Geesthacht (DE)

(21) Appl. No.: 12/300,870

(22) PCT Filed: May 4, 2007
(86) PCT No.: PCT/EP2007/054328

§ 371 Date: May 14, 2009

(30) Foreign Application Priority Data
May 15, 2006 (DE) 10 2006 023 365.4

(51) Int. Cl. A61K 47/42; C08G 63/91; C07K 11/00; A61F 2/02; C12N 5/00

(52) U.S. Cl. 424/423; 525/54.1; 530/317; 514/772.3; 435/395

(57) ABSTRACT

The invention is directed to a multiblock copolymer with shape memory properties and a synthetic precursor of the multiblock copolymer 1. The multiblock copolymer contains: (i) a poly(depsipeptide) segment with an average molecular weight M_{W} in the range of 1,000 to 20,000 g/mol; and (ii) a poly(ε-caprolactone) segment with an average molecular weight M_{W} in the range of 1,000 to 10,000 g/mol.

The invention is directed to a multiblock copolymer with shape memory properties and a synthetic precursor of the multiblock copolymer.

### BACKGROUND OF THE INVENTION AND RELATED ART

Shape memory materials are those that can change their outward shape under the influence of an external stimulus. The present invention is concerned with thermosensitive shape memory plastics, also known as shape memory polymers. The shape memory effect is not a specific material characteristic of polymers; rather, it is a direct result of the combination of polymer structure and polymer morphology and techniques of processing and programming.

In elastomers, a shape memory functionality is achieved when the elastomer is stabilized in its deformed state within a particular temperature range. This can be achieved, for example, by using chain segments as molecular switches. One possibility for a switch function is a thermal transition (T_{Trans}) in the chain segment within the temperature range of interest for the application. If the temperature is greater than T_{Trans} of the switching segment, the segments are flexible and the polymer can be elastically deformed. The temporary shape is fixed by cooling below T_{Trans}. When the polymer is heated again, the permanent shape is restored.

The field of biomedicine is an important area of application for shape memory polymers. In the last 30 years, synthetic, degradable implant materials have ushered in decisive advantages in a wide variety of therapies. Degradable implant materials include, for example, polyhydroxy acids such as polyglycolide or the copolyesters of L-lactic acid and glycolic acid. The appeal of degradable shape memory polymers could also be increased through their use as degradable implant materials; they offer a great potential for application in minimally invasive medicine. Degradable implants could be introduced into the body, for example, in a compressed (temporary) shape through a small incision and could adopt their stored, application-relevant shape when heated to body temperature. After a given time, the implant disintegrates; there is no need for a second operation for removal of the implant.

It is precisely in an application of this kind that the risk of a toxic effect of the shape memory material and its degradation products is significant; these degradation products should be biocompatible.

In this connection, poly(ε-caprolactone)diols with melting temperatures between 46° C. and 64° C. and amorphous copolyesters of diglycolides with glass transition temperatures in the range of 35° C. to 50° C. are described as suitable switching segments for degradable shape memory polymers. The known switching segments have an average molecular weight M_{W} between 500 and 10,000 and a thermal transition of the switching segments in the range between room temperature and body temperature which is favorable for biomedical applications.

A biocompatible and, at the same time, biodegradable multiblock copolymer with shape memory properties can be obtained from crystallizable hard segments of poly(p-dioxanone) and an amorphous switching segment such as a crystallizable poly(ε-caprolactone) segment. The thermoplastic elastomers are produced by means of the co-condensation of two different macrodiols with a difunctional crosslinking unit (for example, diisocyanate, diacid dichloride, or phosgene). To obtain the desired mechanical characteristics, it is very important to achieve high molecular weights M_{W} in the range of 100,000 g/mol. Molecular parameters of this polymer system are the molecular weight, microstructure (sequence), comonomer ratio of the macrodiols, and the hard segment proportion in the multiblock copolymer.

In spite of the above-described advances in the field, there is still a substantial need for shape memory polymers which are hydrolytically degradable in the body, whose degradation products are safe in toxicological respects, and which further have advantageous properties for the planned purpose such as, for example, switching temperatures in the range from 30° C. to 60° C. and processing temperatures of up to 200° C. in biomedical applications.

Therefore, it is the object of the present invention to provide novel biodegradable shape memory materials which have improved or at least equivalent properties in comparison to the known materials.

### Inventive Solution

According to a first aspect of the invention, the above-stated object is met by the multiblock copolymer with shape memory properties according to claim 1. The multiblock copolymer according to the invention contains:
(i) a poly(depsipeptide) segment with an average molecular weight M_{W} in the range of 1,000 to 20,000 g/mol; and
(ii) a poly(ε-caprolactone) segment with an average molecular weight M_{W} in the range of 1,000 to 10,000 g/mol.

The linear multiblock copolymer according to the invention is distinguished by the presence of a poly(depsipeptide) segment that is hydrolytically degradable, and in that the degradation products, namely, amino acids and hydroxy acids, are well-tolerated biologically. The amino acids occurring by hydrolytic degradation are capable of acting as acid/base buffers and accordingly buffer the acidity of the hydroxy acids occurring in the hydrolytic degradation. This mechanism could present a possibility for favorably influencing the course of the healing of wounds, because the release of acid degradation products generally intensifies the occurring inflammation processes. Also, the formation of cationic surface charges in polymers with poly(depsipeptide) blocks during the hydrolytic degradation could be used specifically to moderate the wound healing process. The poly(depsipeptide) segment can act as a hard segment and/or switching segment in the multiblock copolymer. When used as a switching segment, the glass transition temperature (usually in the temperature range between 40° C. and 60° C.) of the amorphous component of the phase determined by the poly(depsipeptide) segment is used as switching temperature. The combination of poly(depsipeptide) segments and poly(ε-caprolactone) segments in multiblock copolymers delivers a hydrolytically degradable thermoplastic elastomer with shape memory properties and switching temperatures in the range of 30° C. to 90° C. and processing temperatures of up to 200° C. in case of blocks which form hard segments and which are based on leucine and diglycolide. Hard segments and switching segments are both formed so as to be hydrolytically degradable.

The indicated molecular weights are to be determined by gel permeation chromatography (GPC). The determination can be carried out in a supplementary manner based on the ¹H NMR spectrum.

A poly(depsipeptide) segment of the following formula (1) is preferred:

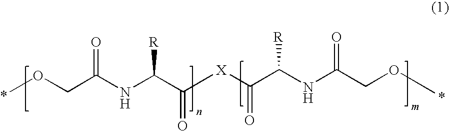

where X is a bridge selected from the group:

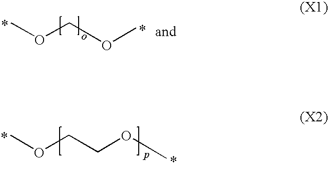

where o=2-20 and p=1-10;
R represents a group selected from H or a branched or unbranched C₁-C₁₀ alkyl radical; and
n and m are given such that the poly(depsipeptide) segment has an average molecular weight M_{W} in the range of 1,000 to 20,000 g/mol.

Further, X preferably represents

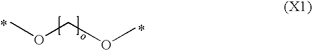

where o=8; or represents

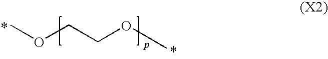

where p=1. Poly(depsipeptide) segments with the variants of the central bridge element (starter) mentioned above can easily be synthesized and, based on first trials, have favorable material characteristics for application in the field of medical engineering.

Further, it is preferable when R in formula (1) represents H, methyl, 1-methylethyl, 2-methylpropyl, or 1-methylpropyl. Accordingly, on the one hand, synthesis rules, known per se, for ring-opening polymerization of morpholine-2,5-dione derivatives with a corresponding bridge-building diol as starter can be drawn upon in the production of the poly(depsipeptide) segments. On the other hand, the generated monomer units in the poly(depsipeptide) segment correspond to the natural amino acids glycine, alanine, valine, leucine and isoleucine, so that a high biocompatibility of the polymer and its degradation products can be expected.

Further—particularly also in combination with each of the above-mentioned variations in the poly(depsipeptide) segment—the poly(ε-caprolactone) segment of formula (2) is preferably:

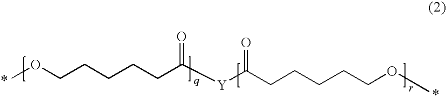

wherein Y represents

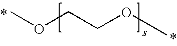

where s=1-10; and
q and r are given such that the poly(ε-caprolactone) segment has an average molecular weight M_{W} in the range of 1,000 to 10,000 g/mol. Preferably, s=2.

The poly(depsipeptide) segments and poly(ε-caprolactone) segments in the multiblock copolymer are preferably coupled by bridges of formulas (3a) and/or (3b):

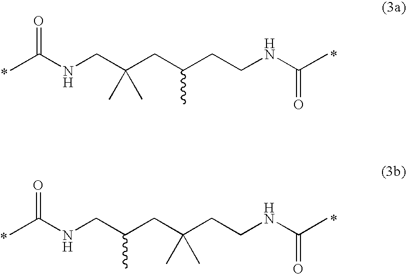

Further, it is preferable when a weight ratio of the poly(depsipeptide) segments to the poly(ε-caprolactone) segments is in the range of 1:1 to 1:10.

Finally, it is preferable when an average molecular weight M_{W} of the multiblock copolymer is in the range of 10,000 to 100,000 g/mol.

A second aspect of the invention is directed to the poly(depsipeptide) of formula (4) which occurs as an intermediate product of the synthesis:

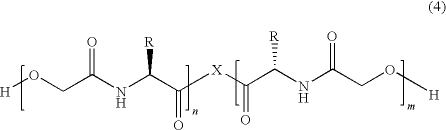

wherein X is a bridge selected from the group:

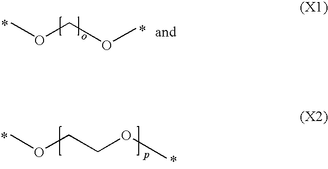

where o=2-20 and p=1-10;
R represents a group selected from H or a branched or unbranched C₁-C₁₀-alkyl radical; and
n and m are given such that the poly(depsipeptide) has an average molecular weight M_{W} in the range of 1,000 to 20,000 g/mol. With respect to preferred variants of the poly(depsipeptides) according to the invention, reference is had to the preferred embodiment forms of bridges X1 and X2 and radical R described above referring to the multiblock polymer.

A third aspect of the invention consists in the use of the multiblock copolymer according to the invention of the type described above as an implant material, as a polymer matrix for the controlled release of active ingredients (active ingredient depots and coatings for encapsulating active ingredients) and as a material for producing framework structures and leading structures (polymer scaffolds and alloplastic scaffolds) for tissue engineering.

The invention will be described more fully in the following with reference to embodiment examples and accompanying drawings.

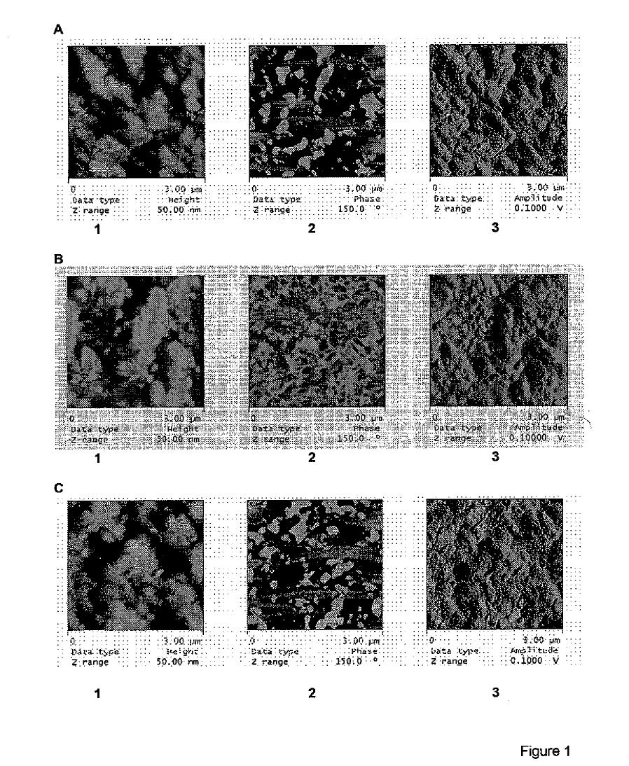

FIG. 1 shows AFM recordings of a surface from a PCL/PIBMD multiblock copolymer at different temperatures;

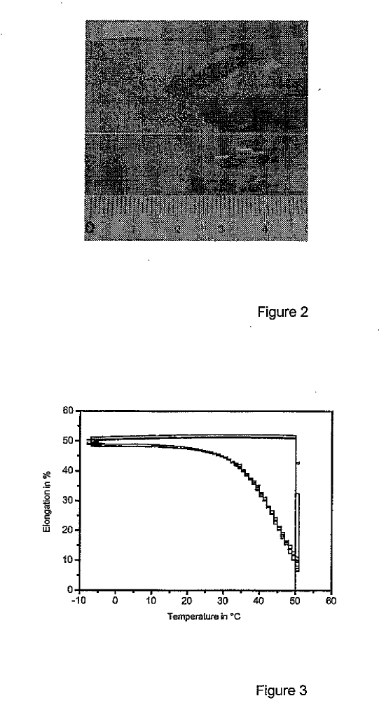

FIG. 2 is a series of photographs illustrating the macroscopic memory effect in the multiblock copolymer (PCL/PIBMD); and

FIG. 3 shows a cyclic, thermomechanical tensile/elongation test of the multiblock copolymer (PCL/PIBMD).

### Synthesis of poly(depsipeptides)

Poly(depsipeptides) are alternating copolymers of α-amino acids and α-hydroxy acids. Different combinations of α-amino acids (for example, L-leucine, L-valine, glycine, L-lysine or L-glutamic acid) and an α-hydroxy acid (glycolic acid, L,L-dilactide or rac-dilactide) can be converted into new materials of a nontoxic and biodegradable nature. A known synthetic approach to poly(depsipeptides) is ring-opening polymerization of morpholine-2,5-dione derivatives in the presence of tin dioctanoate (Sn(oct)₂) as catalyst. Further, an enzymatically catalyzed ring-opening polymerization of morpholine-2,5-diones has been reported. Further, block copolymers of 3(S)-isopropyl-morpholine-2,5-dione and polyethylene oxide (PEO) which are accessible through ring-opening polymerization are known.

Poly(α-hydroxyalkonates) such as poly(L-lactides) or copolymers of L,L-dilactides and diglycolides are used as resorbable implant materials, biodegradable suture material and matrixes for a controlled release of active ingredients. Poly(ε-caprolactone) blocks and poly(p-dioxanone) blocks forming thermoplastic multiblock copolymers with semicrystalline phases and AB polymer networks based on semicrystalline poly(ε-caprolactone) chain segments have been described as biodegradable memory polymers (A. Lendlein et al., Proc. Natl. Acad. Sci. USA 2001, 98(3), 842; A. Lendlein et al., Science 2002, 296(5573), 1673). Biodegradable amorphous poly[(rac-lactide(-ran-glycolide]-urethane networks with shape memory properties have been synthesized by coupling with star-shaped oligomers using an isomeric mixture of 2,2,4- and 2,4,4-trimethyl hexamethylene diisocyanate (TMDI) (A. Lendlein et al. Angew. Chem. 2005, 117, 1212).

### Synthesis of Poly(3(S)-isobutylmorpholine-2,5-dione)diol (PIBMD)

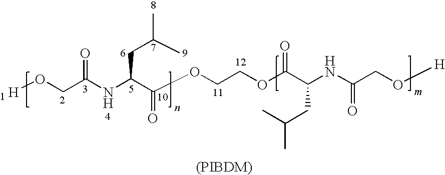

The polymerization was carried out in a dry glass flask with a stirrer. The flask was heated to 50° C., evacuated, and rinsed with dry nitrogen. The flask was charged with 31.3 g of 3(S)-isobutylmorpholine-2,5-dione (IBMD), 0.349 mL of ethylene glycol, and 4 ml of a 0.3-molar Sn(oct)₂ solution. The flask was then evacuated and rinsed repeatedly with dry nitrogen. The reaction mixture was left under nitrogen and heated to 140° C. in an oil bath. After 24 hours, the flask was removed from the oil bath and cooled to room temperature. The product was dissolved in 100 mL DMF and precipitated in 1L diethyl ether. The obtained polymer was collected and dried under vacuum at room temperature for 24 hours. Yield: 80%.

¹H NMR (300 MHz, DMSO): δ=0.80-0.90 ppm (2 d, 6H, CH₃ 8 and 9), 1.45-1.80 ppm (m, 3H, CH 7 and CH₂ 6), 4.20-4.30 ppm (CH₂ 2 in the end group), 4.30-4.50 ppm (CH 5), 4.50-4.73 ppm (AB system, ^{AB}J=14.6 Hz, 2H, CH₂, 2, isot.), 5.49-5.55 ppm (t, ³J=5.8 Hz 1H, OH 1), 8.30-8.40 ppm (d, ³J=7.7 Hz 1H, NH 4); starter: δ=3.80-3.90 ppm (d, ³J=5.7 Hz, 4H, CH₂ 11 and 12).

¹³C NMR (75.41 MHz, DMSO): δ=21.1 ppm (CH₃ 8 or 9), 22.8 ppm (CH₃ 8 or 9), 24.1 ppm (CH 7), 40.4 ppm (CH₂ 6), 49.9 ppm (CH 5), 62.1 (CH₂ 2), 166.6 ppm (COO 10), 171.7 ppm (CONH 3, syndiot.), 171.8 ppm (CONH 3, isot.), 172.3 (CONH 3 end group); starter: δ=61.2 ppm (CH₂ 11 and 12).

Mₙ=6,300 g/mol (¹H NMR), 5,700 g/mol (determination of OH number).

### Synthesis of Poly(3(S)-sec-butylmorpholine-2,5-dione)diol (PBMD)

Production is carried out by a method analogous to that for producing PIBMD, but with the starting materials 3(S)-sec-butylmorpholine-2,5-dione and 1,8-octanediol.

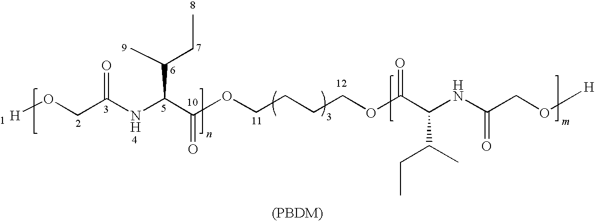

¹H NMR (300 MHz, CDCl₃): δ=0.90-1.09 ppm (2 d, 6H, CH₃ 8 and 9), 1.21-1.75 ppm (m, 2H, CH₂ 7), 1.96-2.04 ppm (m, 1H, CH 6), 4.10-4.20 ppm (CH₂ 2 end group), 4.24-4.30 ppm (m, 1H, CH 5), 4.43-4.90 ppm (AB system, ^{AB}J=14.6 Hz, 2H, CH₂, 2, isot.), 7.50-7.70 ppm (1H, NH 4); starter: δ=4.05-4.10 ppm (4H, CH₂ 11 and 12).

### Synthesis of poly(3-methylmorpholine-2,5-dione)diol (PMMD)

Production is carried out by a method analogous to that for producing PIBMD, but with the starter materials 3-methylmorpholine-2,5-dione and 1,8-octanediol.

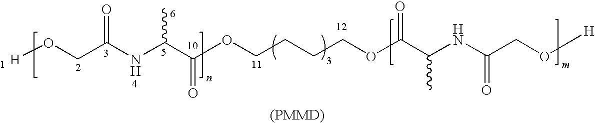

¹H NMR (300 MHz, DMSO): δ=1.2-1.4 ppm (d, 3H, CH₃ 6), 4.3-4.4 ppm (m, 1H, CH 5), 4.5-4.7 ppm (m, 2H, CH₂ 2), 8.3-8.5 ppm (2 d, 1H, NH 4); starter: δ=3.8-3.9 ppm (m, 4H, CH₂ 11 and 12).

Table 1 shows selected properties of the PIBMD, PBMD and PMMD polymers.

TABLE 1

| | Yield | | | | | Tₘ⁵⁾ | ΔH⁵⁾ in | T_{g}⁵⁾ in |
|---|---|---|---|---|---|---|---|---|
| Polymer | in % | M_{OH}¹⁾ | M_{NMR}²⁾ | M_{n,GPC}³⁾ | D⁴⁾ | in ° C. | J/g | ° C. |
| PIBMD | 80 | 5700 | 6300 | 3300 | 2.19 | 170 | 20.3 | 43 |
| PBMD | 80 | 2730 | 1900 | 1800 | 2.71 | 80 | 24.3 | 50 |
| PMMD | 88 | 5600 | 3100 | 10500 | 1.16 | 114 | 24.5 | 62 |

¹⁾Molecular weight by determination of the OH number.
²⁾Molecular weight based on ¹H NMR spectrum.
³⁾Molecular weight GPC.
⁴⁾Molecular weight distribution GPC.
⁵⁾Differential scanning calorimetry (DSC).

### Synthesis of the PCL/PIBMD Multiblock Copolymer

A mixture of 24.0 g (12 mmol) of PCL (poly(ε-caprolactone); trade name CAPA2304 by Solvay Caprolactones, UK; average molecular weight M_{W} 3000 g/mol, 22.5 g (4 mmol) of PIBMD, 16 mmol of TMDI, 43 μL of dibutyl tin laurate (approximately 0.1 percent by weight) and 110 g of N-methylpyrrolidone were added to a two-neck round bottom flask under nitrogen accompanied by continuous stirring by means of a magnetic stirrer. Heating was carried out to a temperature of 80° C. and, after 24 hours, the reaction mixture was analyzed by IR spectroscopy and gel permeation chromatography (GPC). After the NCO bands disappeared in IR at 2270 cm⁻¹, 100 μL of TMDI were added and stirring was carried out for another 24 hours. Subsequently, the reaction mixture was precipitated with 200 mL of 1,2-dichloroethane and with a tenfold excess of diethyl ether. The precipitated multiblock copolymer was collected by filtration and dried under vacuum at room temperature for 24 hours. Yield: 90%.

¹H NMR (300 MHz, DMSO): PIBMD block: δ=0.80-0.90 ppm (2 d, 6H, CH₃ 8 and 9), 1.45-1.80 ppm (m, 3H, CH and CH₂), 4.30-4.50 ppm (CH), 4.50-4.73 ppm (AB system, ^{AB}J=14.6 Hz, 2H, CH₂, isot.), 8.30-8.40 ppm (d, ³J=7.7 Hz 1H, NH); starter: δ=3.82-3.90 ppm (d, ³J=5.7 Hz, 4H, CH₂); PCL block: δ=1.23-1.37 ppm (m, 2H, CH₂), 1.46-1.71 ppm (m, 4H, CH₂, overlapping with PIBMD block), 2.23-2.30 ppm (t, ³J=7.3 Hz 2H, CH₂), 3.94-4.01 ppm (t, ³J=6.6 Hz 2H, CH₂); starter: δ=3.57-3.62 ppm (m, 4H, CH₂) and 4.08-4.13 ppm (m, CH₂); TMDI: δ=0.76-0.93 ppm (m, CH₃, overlapping with PIBMD block), 1.05-1.19 ppm (m, CH₂ and CH), 2.68-3.02 ppm (m, CH₂).

### Synthesis of the PCL/PMMD Multiblock Copolymer

Production was carried out by a method analogous to that used for the production of PCL/PIBMD.

¹H NMR (300 MHz, CDCl₃): PMMD block: δ=1.3-1.4 ppm (CH₃), 4.3-4.4 ppm (s, CH), 4.5-4.7 ppm (CH₂), 7.6-8.0 ppm (NH); starter: δ=3.6 ppm (CH₂). PCL block: δ=1.4-1.5 ppm (m, CH₂ overlapping with PMMD block), 1.5-1.7 ppm (m, CH₂), 2.2-2.40 ppm (2H, CH₂), 4.0-4.1 ppm (2H, CH₂); starter: δ=3.6-3.7 ppm (m, 4H, CH₂) and 4.2 ppm (m, CH₂). TMDI: δ=0.80-0.90 ppm (m, CH₃), 0.9-1.0 ppm (m, CH₂ and CH), 2.8-3.2 ppm (m, CH₂).

Table 2 shows selected properties of the PCL/PIBMD and PCL/PMMD multiblock copolymers.

TABLE 2

| Multiblock- | Poly(depsi- | | | Tₘ₁³⁾ | ΔH₁⁴⁾ | Tₘ₂⁵⁾ | ΔH₂⁶⁾ | T_{g}⁷⁾ |
|---|---|---|---|---|---|---|---|---|
| copolymer | peptide) % by wt. | M_{n,GPC}¹⁾ | D²⁾ | [° C.] | [J/g] | [° C.] | [J/g] | [° C.] |
| PCL/ | 50 | 62000 | 1.65 | 37.9 | 53.2 | 170 | 47.8 | −60 |
| PIBMD | | | | | | | | |
| PCL/ | 50 | 23000 | 1.47 | 44 | 59.4 | 80 | 72 | 28 |
| PMMD | | | | | | | | and |
| | | | | | | | | 51⁸⁾ |

¹⁾Molecular weight GPC.
²⁾Molecular weight distribution GPC.
³⁾First peak in DSC chart.
⁴⁾Enthalpy of the first peak in the DSC chart.
⁵⁾Second peak in the DSC chart.
⁶⁾Enthalpy of the second peak in the DSC chart.
⁷⁾Glass transition temperature from DSC, first run.
⁸⁾Glass transition temperature from DSC, second run.

### Production of a Sample Film

A film with a thickness of 400 μm was produced from the multiblock copolymer PCL/PIBMD by compression melting at 180° C. and 90 bar. The DSC of the PCL/PIBMD film shows that the enthalpy of the PIBMD blocks was very low. The PIBMD blocks must have a high crystallinity to fix the permanent shape of the film. In order to increase the crystallinity of the PIBMD blocks, the film was tempered at 100° C. for 30 minutes and at 80° C. for 24 hours and was then gradually cooled to room temperature.

The PCL/PIBMD multiblock copolymer built from poly(ε-caprolactone) blocks (PCL blocks) and PIBMD blocks was synthesized using 2,2,4- and 2,4,4-trimethyl hexamethylene diisocyanate (TMDI) as coupling reagent.

The phase determined by the PCL blocks with a melting temperature of about 37° C. functions as a switching segment, while the crystalline phase with the higher melting temperature determined by the PIBMD blocks represents the hard segment. The topography and the phase behavior of the multiblock copolymer were analyzed using scanning force microscopy (AFM) based on a polymer film applied to a silicone substrate. The surface topographies of the samples were examined at room temperature to detect the surface morphology above the PCL melting temperature. Subsequently, the samples were cooled again to room temperature.

FIG. 1 shows AFM recordings of the PCL/PIBMD surface at different temperatures, namely, A=room temperature, B=60° C., C=room temperature after cooling from 60° C. View 1 shows the surface topography. View 2 shows the phase, and view 3 shows the amplitude. In view 2, the dark area corresponds to the hard segment and the light area corresponds to the switching segment.

The PCL domains had an expansion of up to 400 nm, whereas PIBMD blocks were present in continuous phase. The comparison of the topography and the phase view shows that the phase domain does not influence the topography of the film. After cooling to room temperature, the PCL phase recrystallized and the AFM photograph resembled the photographs prior to heating. Accordingly, the PCL/PIBMD multiblock copolymer shows a microphase separation between the PCL phase and the PIBMD phase, which leads to the formation of a kind of nano-composite between the PCL blocks determining the switching segment and the PIBMD blocks determining the hard segment.

### Thermal Properties of PCL-PIBMD

A thermal analysis by means of DSC measurement of the multiblock copolymers confirmed that these multiblock copolymers are semicrystalline. PCL diol 3K had a double melting point at 48° C. and 50° C. (ΔH=60.5 J/g) and a glass transition at about −60° C. PIBMD 5K had a melting temperature of about 170° C. (ΔH=20.3 J/g) and a glass transition at 43° C. The two melting temperatures in the multiblock copolymer were 170° C. and 34° C. for the PIBMD blocks and PCL blocks, respectively. The crystalline PIBMD phase prevents a crystallization of the PCL blocks. In the second heating process, PIBMD recrystallized at about 101° C. and showed a melt transition at 170° C. (39.4 J/g), while the PCL phase had a melting temperature of 37° C. (3.0 J/g).

### Mechanical Properties of the PCL/PIBMD Multiblock Copolymer

The mechanical properties of the PCL/PIBMD multiblock copolymers were analyzed by means of tensile/elongation tests above and below Tₘ of the PCL blocks. The results of these tests are compiled in Table 3.

TABLE 3

| E^{[a]} | σ_{b}¹⁾ | ε_{b}¹⁾ | E²⁾ | σ_{b}²⁾ | ε_{b}²⁾ | εₘ | R_{f}(1) | Rᵣ(1) | R̅^{f,2-5} | R̅^{r,2-5} |
|---|---|---|---|---|---|---|---|---|---|---|
| [MPa] | [MPa] | [%] | [MPa] | [MPa] | [%] | [%] | [%] | [%] | [%] | [%] |
| 72 ± 12 | 14.5 ± 2.1 | 420 ± 80 | 30.4 ± 9.0 | 2.8 ± 0.1 | 70 ± 11 | 50 | 97.8 | 97.1 | 96.3 | 98.8 |

¹⁾Determined at 25° C.
²⁾Determined at 75° C.

### Shape Memory Properties of PCL/PIBMD

A PCL/PIBMD multiblock copolymer in its permanent shape as a helically twisted strip was changed from its permanent shape at high temperature (T=120° C.) into the temporary shape (flat polymer strip). The deformed shape was fixed by cooling to room temperature. In order to restore the permanent shape, the sample was heated above the switching temperature T_{Trans} (to about 60° C.) and the original permanent shape was restored. The macroscopic shape memory effect of PCL-PIBMD is shown in FIG. 2.

The shape memory properties of the PCL-PIBMD multiblock copolymer were quantified by means of cyclical thermomechanical examinations, wherein a maximum elongation of εₘ=50% was applied. The measurement results of five successive thermocycles are shown in FIG. 3. The superposition of the curves (N=2-5) indicates that the shape memory properties adopt constant values after passing through the first cycle (N=1) so that significant relaxation effects or the occurrence of irreversible effects during the thermomechanical examination can be ruled out.

1. Multiblock copolymer with shape memory properties, containing:
(i) a poly(depsipeptide) segment with an average molecular weight M_{W} in the range of 1,000 to 20,000 g/mol; and
(ii) a poly(ε-caprolactone) segment with an average molecular weight M_{W} in the range of 1,000 to 10,000 g/mol.

2. Multiblock copolymer according to claim 1, with a poly(depsipeptide) segment of formula (1): 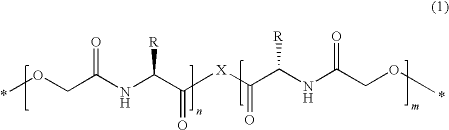
where X is a bridge selected from the group: 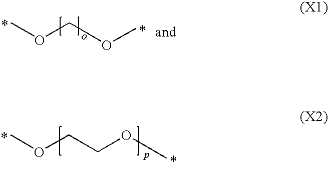
where o=2-20 and p=1-10;
R represents a group selected from H or a branched or unbranched C₁-C₁₀ alkyl radical; and
n and m are given such that the poly(depsipeptide) segment has an average molecular weight M_{W} in the range of 1,000 to 20,000 g/mol.

3. Multiblock copolymer according to claim 2, wherein X represents 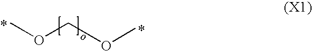
where o=8.

4. Multiblock copolymer according to claim 2, wherein X represents 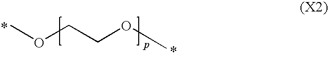
and p=1.

5. Multiblock copolymer according to claim 2, wherein R represents H, methyl, 1-methylethyl, 2-methylpropyl, or 1-methylpropyl.

6. Multiblock copolymer according to claim 1, with a poly(ε-caprolactone) segment of formula (2): 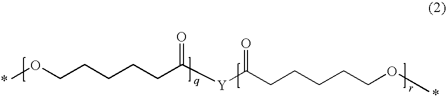
wherein Y represents 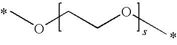
where s=1-10; and
q and r are given such that the poly(ε-caprolactone) segment has an average molecular weight M_{W} in the range of 1,000 to 10,000 g/mol.

7. Multiblock copolymer according to claim 6, wherein s=2.

8. Multiblock copolymer according to claim 2, wherein the poly(depsipeptide) segments and poly(ε-caprolactone) segments in the multiblock copolymer are coupled by bridges of formulas (3a) and/or (3b):

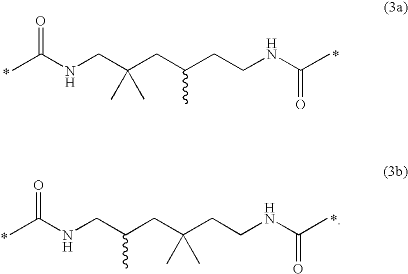

9. Multiblock copolymer according to claim 2, wherein a weight ratio of the poly(depsipeptide) segments to the poly(ε-caprolactone) segments is in the range of 1:1 to 1:10.

10. Multiblock copolymer according to claim 1, with an average molecular weight M_{W} of the multiblock copolymer in the range of 10,000 to 100,000 g/mol.

11. Poly(depsipeptide) of formula (4): 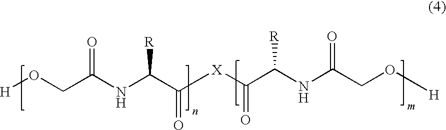
wherein X is a bridge selected from the group: 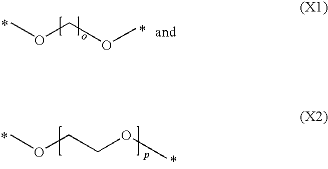
where o=2-20 and p=1-10;
R represents a group selected from H or a branched or unbranched C₁-C₁₀-alkyl radical; and
n and m are given such that the poly(depsipeptide) has an average molecular weight M_{W} in the range of 1,000 to 20,000 g/mol.

12. (canceled)

13. A method for introducing an implant into a subject comprising the step of implanting into the subject the multiblock copyolymer according to claim 1.

14. A method for controlling the release of an active ingredient comprising (i) depositing the ingredient in a polymer matrix (active ingredient depot) comprising the multiblock copolymer according to claim 1, or (ii) coating or encapsulating the active ingredient with a polymer matrix comprising the multiblock copolymer according to claim 1.

15. A method for engineering tissue comprising producing a framework structure and/or leading structure (polymer scaffold and/or alloplastic scaffold) using the multiblock copolymer according to claim 1.
